(19) United States
(12) Patent Application Publication
(10) Pub. No.: US 2024/0197972 A1
(43) Pub. Date: Jun. 20, 2024

(54) PRIMING METHOD AND SYSTEM FOR FORWARD OSMOSIS

(71) Applicant: Gambro Lundia AB, Lund (SE)

(72) Inventors: Christian Vartia, Veberöd (SE); Henrik Lindgren, Genarp (SE)

(21) Appl. No.: 18/554,437

(22) PCT Filed: Apr. 5, 2022
(86) PCT No.: PCT/EP2022/058923

§ 371 Date: Oct. 6, 2023

Related U.S. Application Data
(60) Provisional application No. 63/172,850, filed on Apr. 9, 2021.

(30) Foreign Application Priority Data
Dec. 21, 2021 (SE) 2151562-2

(51) Int. Cl. A61M 1/16; A61M 1/26; B01D 61/00

(52) U.S. Cl. CPC A61M 1/1658; A61M 1/1672; A61M 1/1694; A61M 1/267; B01D 61/0022; B01D 61/0023; B01D 61/0024; A61M 2205/3382; B01D 2311/02; B01D 2313/243

(57) ABSTRACT

Provided herein is an apparatus for producing dialysis fluid including a forward osmosis unit used for diluting a dialysis concentrate for producing dialysis fluid. The FO-unit includes a FO-membrane that separates a first side from a second side. The apparatus also comprises a first flow path including a first side and a control arrangement providing priming fluid from a source to the first flow path. A return path fluidly connects an inlet port of the first side to an outlet port of the first side to allow priming fluid from the outlet port to circulate to the inlet port via the return path. The apparatus further comprises a gas collection chamber arranged in the first flow path between the first side and the return path. The gas collection chamber removes gas from first flow path. The present disclosure also relates to a method for priming a FO-unit.

### PRIORITY CLAIM

This application claims priority to and the benefit of U.S. Provisional Application No. 63/172,850, filed Apr. 9, 2021, entitled, Priming, Method and System for Forward Osmosis Unit, and the Swedish Patent Application No. 2151562-2, filed Dec. 21, 2021, entitled, Priming of a Forward Osmosis Unit, the entire contents of which is incorporated herein by reference and relied upon.

### TECHNICAL FIELD

The present invention relates to the field of priming, and in particular the priming of a forward osmosis unit arranged for producing dialysis fluid.

### BACKGROUND

Kidney failure occurs when your kidneys lose the ability to sufficiently filter waste from the patient's blood. The waste accumulates in the body, which with time becomes overloaded with toxins. Kidney failure can be life threatening if left untreated. Reduced kidney function and, above all, kidney failure is treated with dialysis. Dialysis removes waste, toxins and excess water from the body that normal functioning kidneys would otherwise remove.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysis fluid to cause diffusion. HD fluids are typically created by the dialysis machines by mixing concentrates and clean water.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is delivered directly to the extracorporeal circuit, providing convective clearance. Here, more fluid than the patient's excess fluid is removed from the patient, causing the increased convective transport of waste products from the patient. The additional fluid removed is replaced via the substitution or replacement fluid.

Another type of kidney failure therapy is peritoneal dialysis (“PD”), which infuses a dialysis solution, also called dialysis fluid, into a patient's peritoneal cavity via a catheter. The dialysis fluid is in contact with the peritoneal membrane located in the patient's peritoneal cavity. Waste, toxins and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated, e.g., multiple times. PD fluids are typically prepared in a factory and shipped to the patient's home in ready-to-use bags.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment, where fluid transport is driven by gravity. If initially full of spent dialysis fluid, the patient manually connects an implanted catheter to a drain to allow the used or spent dialysis fluid to drain from the patient's peritoneal cavity. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal cavity, wherein the transfer of waste, toxins and excess water takes place. After a dwell period, the patient repeats the manual dialysis procedure, for example, four times per day. If the patient is not initially full of spent dialysis fluid, the sequence is instead a patient fill, dwell and drain. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly via a patient line to the patient's implanted catheter, to a source or bag of fresh dialysis fluid and to a fluid drain. APD machines pump fresh dialysis fluid from the fresh dialysis fluid source, through the catheter and into the patient's peritoneal cavity. APD machines also allow for the dialysis fluid to dwell within the patient's peritoneal cavity and for the transfer of waste, toxins and excess water to take place. The source may include multiple liters of dialysis fluid, including several solution bags.

Dialysis treatments may be performed at a clinic or remotely such as in the patient's home. Transportation of dialysis fluid adds costs to the treatment and has a negative impact on the environment. The storage of dialysis fluid is space demanding and large dialysis fluid bags need to be handled by the user. A way to reduce or eliminate the amount of dialysis fluid transported to the patient's home is needed accordingly.

### SUMMARY

To reduce the above-identified negative consequences from the transportation of dialysis fluid to the patient's home, dialysis fluid may be produced at the point of care from concentrates. In the apparatus and method of the present disclosure, forward osmosis (“FO”) may be used for diluting a dialysis concentrate with water to provide a diluted dialysis concentrate, which may be referred to as a dialysis solution. The dialysis solution may thereafter be mixed with other concentrates to provide a final dialysis fluid that can be used in a dialysis treatment to treat a patient. The final dialysis fluid may be dialysis fluid for PD, a dialysis fluid for HD or HDF, or a replacement fluid or substitution fluid for HF or HDF. FO makes use of an osmotic pressure gradient between a feed fluid and the concentrate as draw fluid, which are separated with a FO-membrane. The osmotic pressure gradient is used as an energy source for causing water to migrate from the feed fluid to the draw fluid, making FO an attractive low-energy alternative. The FO-membrane is a semipermeable membrane, and typically has a hollow fiber geometry. The FO-membrane of a hollow fiber type has thousands of hollow fibers packed together in a bundle and arranged in a FO-unit. One of the feed fluid and draw fluid is passed inside the fiber lumen (lumen side), while the other fluid is simultaneously passed on the outside of the lumen (shell side). The feed fluid is for example water or spent (used) dialysis fluid. If spent dialysis fluid is used as the feed fluid, the amount of fresh water used in the treatment can be greatly reduced. Alternative FO-membrane types are for example flat sheet or spiral wound membranes.

A varying dilution performance has been seen during tests arising from air present in the FO-unit. Some fibers of the FO-membrane may then remain unused, thereby decreasing the effective area of the FO-membrane. There is thus a need for a method that effectively removes the air present in the FO-unit.

It is an objective of the present disclosure to alleviate at least some of the drawbacks with the prior art. It is a further objective to provide a method for efficiently removing air present in the FO-unit. It is yet a further objective to provide a method for automatically removing air present in the FO-unit.

These objectives and others are at least partly achieved by the apparatus and method according to the independent claims, and by the embodiments according to the dependent claims.

According to one aspect, which may be combined in whole or in part with any other aspect or portion thereof, the present disclosure relates to an apparatus for producing fluid for dialysis. The apparatus comprises a forward osmosis, FO-, unit configured to be used for diluting a dialysis concentrate in a process for producing a dialysis fluid. The FO-unit includes a FO-membrane that separates a first side from a second side of the FO-unit. The apparatus further comprises a first flow path including the first side and a control arrangement configured to provide priming fluid from a priming fluid source to the first flow path. The apparatus further comprises a return path fluidly connecting an inlet port of the first side to an outlet port of the first side of the FO-unit, to allow priming fluid expelled from the outlet port to circulate to the inlet port via the return path. The apparatus further comprises a gas collection chamber arranged in the first flow path between the first side and the return path, wherein the gas collection chamber is configured to receive gas removed from the first flow path.

The apparatus enables efficient priming of the first side by providing a return path where priming fluid residing at the first side can be circulated to a gas collection chamber, and where gas can be accumulated and evacuated. As the priming fluid can be circulated back to the inlet port, it becomes easier to remove gas from the priming fluid as the fluid will contain less and less gas after each pass through the gas collection chamber as opposed to using new priming fluid continuously for the priming. Also, the priming fluid can be reused during the priming so that less priming fluid is wasted.

According to some embodiments, the control arrangement is configured to monitor a level of priming fluid in the gas collection chamber and to provide priming fluid to the gas collection chamber until the level of priming fluid in the gas collection chamber has reached a predetermined level. Gas may thereby be accumulated in the gas collection chamber at the same time that the gas collection chamber contains enough priming fluid to provide a flow of priming fluid out from the gas collection chamber.

According to some embodiments, the apparatus is positioned and arranged to evacuate gas from the gas collection chamber while providing priming fluid to the gas collection chamber. Gas may thereby be removed from the first flow path.

According to some embodiments, the gas collection chamber comprises a gas outlet port for evacuating gas from the gas collection chamber. Gas may thereby be evacuated from the gas collection chamber.

According to some embodiments, the apparatus comprises a gas collection path fluidly connecting the gas outlet port of the gas collection chamber to a drain. Gas may thereby be transported to the drain from the gas collection chamber.

According to some embodiments, the control arrangement is configured to provide priming fluid from the priming fluid source to the first side of the FO-unit via the first flow path and the gas collection chamber, to fill the first side with priming fluid. The first side may thereby be filled with priming fluid from which gas already has been removed, so that resulting deaeration of the first side is supported.

According to some embodiments, the control arrangement is configured to provide priming fluid from the priming fluid source to the first side of the FO-unit via the first flow path and the gas collection chamber, upon the level of priming fluid in the gas collection chamber reaching a predetermined level. Thereby, a sufficient volume of priming fluid in the gas collection chamber is present, such that a flow of priming fluid can pass through the gas collection chamber at the same time as gas is accumulated in the gas collection chamber.

According to some embodiments, the control arrangement is configured to stop flow via the return path while priming fluid is provided to the first flow path from the priming fluid source. The filling of the gas collection chamber and/or the first side may therefore be more easily controlled.

According to some embodiments, the control arrangement is configured to circulate the priming fluid provided to the first side in a first direction in a recirculation path comprising the first side, the return path and the gas collection chamber. Gas bubbles at the first side may thereby be removed and collected in the gas collection chamber.

According to some embodiments, the control arrangement is configured to circulate the priming fluid in a second direction in the recirculation path. Even more gas bubbles at the first side may therefore be collected in the gas collection chamber, as the flow in the other direction may cause other gas bubbles to come loose from the first side versus flowing in the first direction. Also, if the first direction is from the uppermost part (inlet port) to a lowermost part (outlet port) of the first side, and the second direction may be from the lowermost part (outlet port) to the uppermost part (inlet port) of the first side, so that the flow in the first direction may remove gas bubbles from the first side, while the flow in the second direction may transport gas bubbles trapped at the inlet of the first side to the gas collection chamber.

According to some embodiments, the control arrangement is configured to repeat the circulation of the priming fluid in the first direction in the recirculation path, and circulation of the priming fluid in the second direction in the recirculation path, at least one time until one or more predetermined priming criteria is/are fulfilled. More gas bubbles may thereby come loose and be transported to the gas collection chamber, as repeated shifts in direction cause a change in direction of shear force at the first side, which causes more gas bubbles to come loose.

According to some embodiments, the apparatus comprises a second flow path including the second side for providing fluid to the second side, wherein the control arrangement is configured, upon fulfilling the one or more predetermined priming criteria, to provide fluid from a solution source via the second flow path to an inlet port to the second side, the inlet port is arranged below an outlet port of the second side. The second side is therefore also primed. The second side may be primed before, during and/or after the first side is primed.

According to some embodiments, the control arrangement is configured to circulate the priming fluid in the first direction at a first flow rate and to circulate the priming fluid in the second direction at a second flow rate, wherein the first flow rate is different from the second flow rate. A different magnitude of shear forces may therefore be accomplished at the first side. The flow rates may also be different as they are intended to accomplish different things, for example the first flow rate may be intended for loosening gas bubbles from the first side, while the second flow rate may be intended for transporting the loosened gas bubbles to the gas collection chamber.

According to some embodiments, the first flow rate is greater than the second flow rate. For example, in the second direction it might be sufficient to have a slower flow rate if the purpose of the second flow rate is solely transportation of the gas bubbles to the gas collection chamber. Greater first flow rates create a higher shear force at the first side that more easily removes gas bubbles.

According to some embodiments, the control arrangement is configured to circulate the priming fluid in the first direction in the recirculation path for a predetermined first time period and to circulate the priming fluid in the second direction in the recirculation path for a second time period, wherein the first time period and the second time period have different lengths. The priming may thereby be optimized in terms of time, as the effect to be achieved with the flow in either direction may be different and accordingly need more or less time to be achieved.

According to some embodiments, the first time period is greater than the second time period. A longer transportation of the priming fluid from the first side to the gas collection chamber may thereby be achieved during the first time period versus during the second time period.

According to some embodiments, the control arrangement comprises at least one pump. Providing the priming fluid and/or the circulation of the priming fluid may thereby be accomplished by means of the one or more pump(s).

According to some embodiments, the at least one pump comprises a first pump arranged to provide the priming fluid to the first path from the priming fluid source.

According to some embodiments, the at least one pump comprises a second pump arranged to circulate the priming fluid in the recirculation path.

According to some embodiments, the gas collection chamber comprises at least two fluid ports, and wherein the priming fluid is allowed to enter and to be expelled via any of the at least two ports. The priming fluid may thereby be transported in two opposite directions through the gas collection chamber.

According to some embodiments, the priming fluid is the same fluid that is used during production for diluting a dialysis concentrate, and wherein the priming fluid is water or used dialysis solution. The handing of the apparatus during priming may therefore be facilitated as there is no need to prepare the apparatus differently than when for producing a dialysis fluid.

According to a second aspect, which may be combined in whole or in part with any other aspect or portion thereof, the present disclosure relates to a method for priming a forward osmosis, FO-, unit configured to be used for diluting a dialysis concentrate in a process for producing a dialysis fluid. The FO-unit includes a FO-membrane that separates a first side from a second side of the FO-unit. The method comprises: providing priming fluid to a first flow path comprising the first side and a gas collection chamber configured to receive gas removed from the first flow path. The method further comprises circulating the provided priming fluid in a recirculation path comprising the first side, a return path and the gas collection chamber. The return path fluidly connects an inlet port of the first side to an outlet port of the first side of the FO-unit.

According to some embodiments, the providing comprises monitoring a level of priming fluid in the gas collection chamber and providing priming fluid to the gas collection chamber until the level of priming fluid in the gas collection chamber has reached a predetermined level.

According to some embodiments, the method comprises evacuating gas from the gas collection chamber while providing priming fluid to the gas collection chamber.

According to some embodiments, the method comprises evacuating gas from the gas collection chamber to a drain via a gas collection path fluidly connecting a gas outlet port of the gas collection chamber to the drain.

According to some embodiments, the method comprises providing priming fluid from the priming fluid source to the first side of the FO-unit via the first flow path and the gas collection chamber to fill the first side with priming fluid.

According to some embodiments, the method comprises providing priming fluid from the priming fluid source to the first side of the FO-unit via the first flow path and the gas collection chamber, upon the level of priming fluid in the gas collection chamber reaching a predetermined level.

According to some embodiments, the providing comprises stopping flow via the return path while priming fluid is provided to the first flow path from the priming fluid source.

According to some embodiments, the method comprises circulating the priming fluid provided to the first side in a first direction in the recirculation path.

According to some embodiments, the method comprises circulating the priming fluid in a second direction in the recirculation path.

According to some embodiments, the method comprises repeatedly circulating the priming fluid in the first direction in the recirculation path and circulating the priming fluid in the second direction in the recirculation path at least one time until one or more predetermined priming criteria is/are fulfilled.

According to some embodiments, upon fulfilling the one or more predetermined priming criteria, the method includes providing fluid from a solution source via a second flow path to an inlet port to the second side, which is arranged below an outlet port of the second side.

According to some embodiments, the method comprises circulating the priming fluid in the first direction at a first flow rate and circulating the priming fluid in the second direction at a second flow rate, wherein the first flow rate is different from the second flow rate.

According to some embodiments, the first flow rate is greater than the second flow rate.

According to some embodiments, the method comprises circulating the priming fluid in the first direction in the recirculation path for a predetermined first time period and circulating the priming fluid in the second direction in the recirculation path for a second time period, wherein the first time period and the second time period have different lengths.

According to some embodiments, the first time period is greater than the second time period.

According to a third aspect, which may be combined in whole or in part with any other aspect or portion thereof, the present disclosure relates to a computer program comprising instructions to cause the apparatus, according to any one of the embodiments or aspect described herein, to execute the method according to any one of the embodiments or aspects described herein.

According to a fourth aspect, which may be combined in whole or in part with any other aspect or portion thereof, the present disclosure relates to a computer-readable medium having stored thereon the computer program according to the third aspect.

### BRIEF DESCRIPTION OF THE DRAWINGS

FIG. 1 illustrates part of an apparatus for generating a dialysis solution including a FO-unit according to some embodiments of the present disclosure.

FIG. 2 schematically illustrates a gas collection chamber according to some embodiments of the present disclosure.

FIG. 3 is a flow chart illustrating method steps for performing a priming procedure according to some embodiments of the present disclosure.

FIGS. 4A to 4E illustrate a priming sequence for priming the FO-unit in FIGS. 1 and 5 according to some embodiments of the present disclosure.

FIG. 5 illustrates an apparatus for generating a dialysis solution according to some embodiments of the present disclosure.

### DETAILED DESCRIPTION

Continuous efficient water extraction from the feed solution is of high importance in order to timely produce an accurate dialysis solution. Feed solution and draw solution are often available in limited volumes, and care should be taken to use them efficiently. The FO process may also work in a single pass mode, whereby high efficiency of the FO process is important to make full use of the osmotic pressure gradient. If some of the hollow fibers of the hollow fiber FO-membrane cannot be utilized because there is gas, such as air, stopping the solution flow inside the lumen, the effective area of the FO-membrane will be reduced and the FO process will be less effective. This might especially be a problem at the one of the first side and second side, where the fluid during production is introduced from an uppermost part and expelled from a lowermost part of the side. Here, some excess gas in the FO-unit will not follow the flow of the fluid because it will become trapped at the inlet (the uppermost part) as it is lighter than the fluid or because it is trapped inside the lumens due to the narrowness of the lumens. The gas will instead remain in the FO-unit. There is then a risk that the concentrate solution will not be diluted sufficiently, and that the final dialysis solution will not be produced correctly. “Gas” here includes air or any other gas present in the apparatus from start, any air or other gas fed to the FO-unit or any air or other gas resulting from degassing of the fluids present in the apparatus. Alternative FO-membrane types that may be used in the present disclosure are for example flat sheet membranes or spiral wound membranes.

To obviate the above-described situation, an apparatus for producing fluid for dialysis is proposed which is arranged for efficient priming of its FO-unit. Also, methods for efficient priming are disclosed, which can be performed automatically by the apparatus. The apparatus comprises a gas collection chamber and a return path. The gas collection chamber enables gas to be collected and removed from the first side of the FO-unit. The return path connects the outlet of the first side of the FO-unit with the inlet of the same first side of the FO-unit. The FO-unit is arranged such that during production, fluid will be introduced from an uppermost part (inlet port) and expelled from a lowermost part (outlet port) of the first side. The return path is external to the FO-unit. The return path enables priming fluid to recirculate in a recirculation path including the first side of the FO-unit, the return path and the gas collection chamber. Gas can then be transported by the circulating priming fluid and released in the gas collection chamber. In some embodiments, the priming fluid is sequentially pumped in opposite directions to more effectively remove gas being trapped inside the FO-unit, at the first side. The priming fluid can then be pumped at a high flow rate in a first direction (that is the same as the fluid direction during production) to remove gas bubbles from the lumens, and at a lower flow rate in the second direction (opposite the fluid direction during production) to transport removed gas bubbles trapped at the uppermost part of the first side from the first side to the gas collection chamber. Hence, the invention removes gas from the FO-unit, thereby increasing the effective area of the FO-membrane. The water extraction efficiency can thereby be increased and in some embodiments even maximized by making the entire FO-membrane surface area available for water extraction.

References that are the same throughout the figures may not be textually described in each embodiment but nevertheless include, for each embodiment, all of the structure, functionality and alternatives that are described.

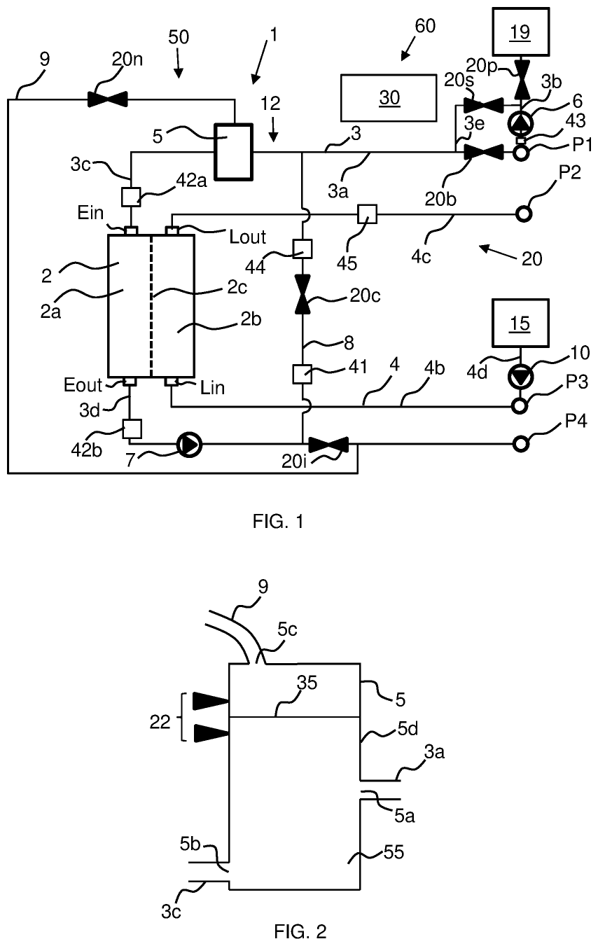

FIG. 1 illustrates a part 50 of an apparatus 1 for producing fluid for dialysis. The part 50 includes components that enable efficient priming of the FO-unit 2. But it is first explained how the part 50 operates in a context of producing fluid for dialysis. The part 50 includes the FO-unit 2, a first flow path 3 and a second flow path 4. The FO-unit 2 includes a FO-membrane 2c that separates a first side 2a from a second side 2b of the FO-unit 2. A “side” of unit 2 may also be referred to herein as a “compartment” or “chamber”. The FO-unit 2 typically includes a cartridge that encloses the first side 2a, second side 2b and FO-membrane 2c. The first flow path 3 is arranged for providing a first fluid to the first side 2a of the FO-unit 2, and for removing the first fluid outputted from the first side 2a. The second flow path 4 is arranged for providing a second fluid to the second side 2b of the FO-unit 2, and for removing the second fluid from the second side 2b. Depending on the compositions of the fluids, one fluid may be referred to as a feed fluid and the other one as a draw fluid. In the FO-process, the feed fluid expels water to the draw fluid because of the osmotic pressure gradient. In some embodiments, during production, feed fluid such as water or spent dialysis fluid is passed to the first side 2a. The first fluid is then the feed fluid, and the first side 2a may be referred to as a feed side. Spent dialysis fluid may also be referred to herein as used dialysis fluid or effluent. The draw fluid such as a dialysis concentrate is passed to the second side 2b. The second fluid is then draw fluid, and the second side 2b may be referred to as a draw side. In the FO-unit 2, water from the feed solution travels through the FO-membrane 2c to the dialysis concentrate and thereby dilutes the dialysis concentrate into a diluted dialysis concentrate solution. This solution may thereafter be mixed with one or more other concentrate(s) to provide a final dialysis fluid. Hence, the FO-unit 2 is configured to be used for diluting a dialysis concentrate in a process for producing a dialysis fluid. The dewatered feed fluid is typically sent to drain.

In the illustrated embodiment of FIG. 1, the FO-unit 2 has an inlet port Eᵢₙ in fluid communication with the first side 2a and through which the first fluid is passed into the first side 2a, and an outlet port Eₒᵤₜ in fluid communication with the first side 2a, wherethrough the first fluid is passed out from the first side 2a. The inlet port Eᵢₙ is arranged above the outlet port Eₒᵤₜ. The FO-unit 2 also has an inlet port Lᵢₙ in fluid communication with the second side 2b through which the second solution is passed into the second side 2b, and an outlet port Lₒᵤₜ in fluid communication with the second side 2b, wherethrough the second solution is passed out from the second side 2b. The outlet port Lₒᵤₜ is arranged above the inlet port Lᵢₙ. To enable efficient priming, the first flow path 3 further comprises a gas collection chamber 5. The gas collection chamber 5 is configured to receive gas removed from the first flow path 3, and its function is explained in more detail in the following with reference to priming.

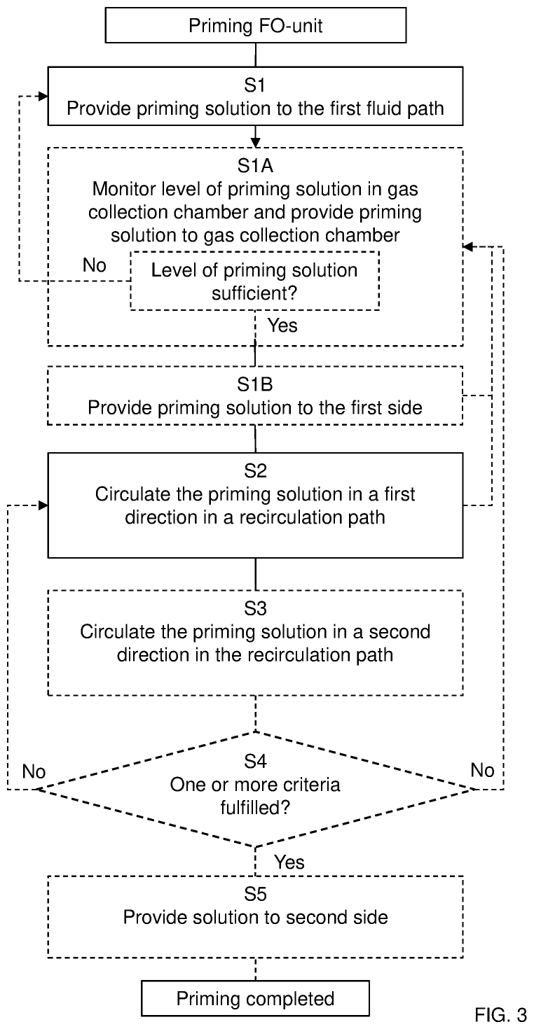

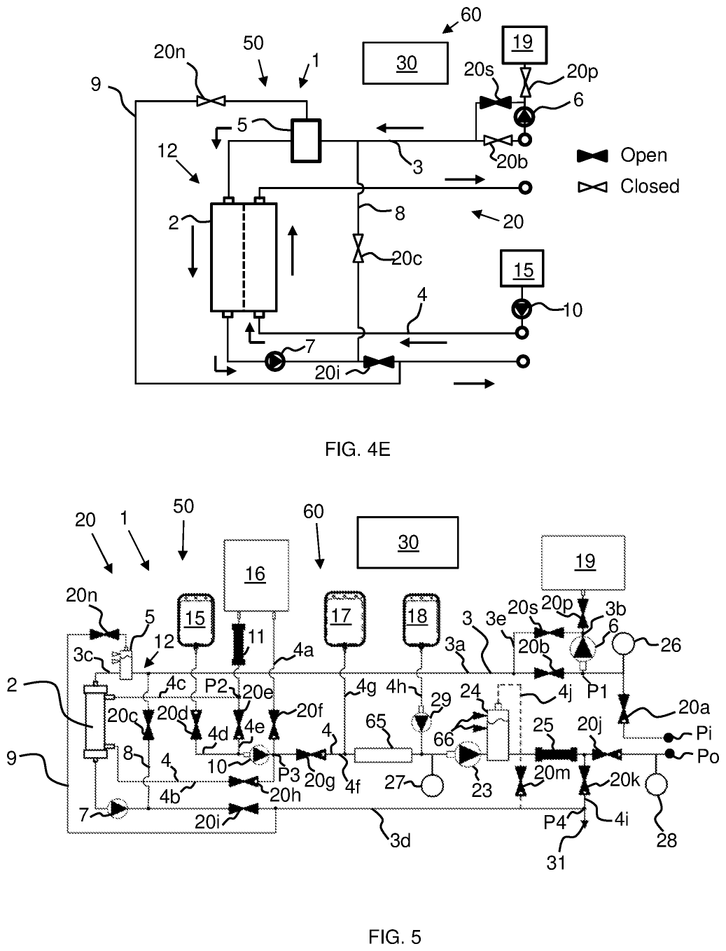

The first flow path 3 comprises a first side input line 3a which is arranged between a point P1 connected to a source of first fluid and an inlet port 5a (FIG. 2) of the gas collection chamber 5. The first side input line 3a fluidly connects the point P1 and the inlet port 5a. A first side input line valve 20b is arranged to operate with the first side input line 3a to regulate the flow in the first side input line 3a. The first flow path 3 further comprises a container line 3b arranged between a container 19 and the point P1, which connects the container 19 and the point P1. A first pump 6 is arranged to operate with the container line 3b, to provide a flow in the container line 3b. A container valve 20p is connected to the container line 3b. A direct flow line 3e is arranged between the container line 3b and the first side input line 3a. Hence, the direct flow line 3e fluidly connects the container line 3b and the first side input line 3a. The direct flow line 3e connects to the container line 3b between the container valve 20p and the first pump 6. A direct flow line valve 20s is connected to the direct flow line 3e. The direct flow line 3e is connected to the first side input line 3a between the first side input valve 20b and the gas collection chamber 5. The container 19 is a source of first fluid and here comprises a first fluid, for example spent dialysis fluid. The spent dialysis fluid has for example previously been pumped from a patient connected at the point P1 to the container 19 using the first pump 6. Alternatively, the point P1 is connected to a source of water, for example a water tap. The first pump 6 may then pump water to the container 19 and store it for later use. In some embodiments, the first pump 6 pumps spent dialysis fluid from the container 19 to the first side input line 3a, gas collection chamber 5, etc., by opening container valve 20p and first side input line valve 20b, closing direct flow line valve 20s and pumping with first pump 6 (in a backward direction). Hence, in some embodiments, the first pump 6 is a bi-directional pump. Spent dialysis fluid or water is then pumped into the first side input line 3a from the container 19 via the container line 3b. The first pump 6 may instead pump spent dialysis fluid directly from a patient or other source, or pump water from tap, connected to the point P1, by pumping with first pump 6 (in a forward direction), opening direct flow line valve 20s and closing container valve 20p and first side input line valve 20b. Spent dialysis fluid or water is then pumped into the first side input line 3a via the container line 3b and the direct flow line 3e. The first pump 6 is for example a volumetric pump, such as a piston pump, that operates in open loop (certain voltage or frequency command from control arrangement 50 to provide a certain flow rate). Alternatively, the first pump 6 is a non-volumetric pump that operates with feedback from a flow rate sensor 43 to reach a certain flow rate. The flow rate sensor 43 as illustrated in FIG. 1 is connected to container line 3b between the first pump 6 and the point P1, but may instead be connected to the container line 3b at any side of the first pump 6, except between the container 19 and the connection point of the direct flow line 3e to the container line 3b. The first flow path 3 further comprises a connection line 3c which is arranged between an outlet port 5b (FIG. 2) of the gas collection chamber 5 and the inlet port Eᵢₙ. Hence, the connection line 3c fluidly connects the outlet port 5b of the gas collection chamber 5 and the inlet port Eᵢₙ. The first pump 6 is arranged to pump fluid from the container 19 or other source at point P1 in the first fluid line 3a and provides the first fluid to the first side 2a via the gas collection chamber 5. The first flow path 3 also includes the first side 2a. The first flow path 3 further comprises a drain line 3d. The drain line 3d is arranged between the outlet port Eₒᵤₜ and a drain point P4, wherefrom the spent first fluid can be passed to drain (Ref. 31, FIG. 5). Hence, the drain line 3d fluidly connects the outlet port Eₒᵤₜ and a drain. A second pump 7 is arranged to operate with the drain line 3d to provide a flow of fluid in the drain line 3d. A drain valve 20i is arranged to regulate flow in the drain line 3d. The drain valve 20i is arranged to operate with the drain line 3d between a connection point of a return path 8 to the drain line 3d and a connection point of the gas collection path 9 to the drain line 3d.

The second flow path 4 comprises a second side input line 4b. The second side input line 4b is arranged between a source of a second fluid at a point P3 and the inlet port Lᵢₙ, and fluidly connects the source of a second fluid and the inlet port Lᵢₙ. In some embodiments, point P3 is an outlet port to a main line 4f (FIG. 5) be able to mix directly from concentrate in concentrate container 15. The second side fluid path 4 further comprises a concentrate line 4d arranged between a concentrate container 15 and the point P3, to connect the concentrate container 15 and the point P3. A concentrate pump 10 is arranged to operate with the concentrate line 4d to provide a flow in the concentrate line 4d. The concentrate container 15 comprises for example a fluid dialysis concentrate. The concentrate pump 10 is positioned and arranged to pump fluid from the concentrate container 15 or other source at point P3 in the second side input line 4b and provide the second fluid, the concentrate fluid, to the second side 2b. The second flow path 4 also includes the second side 2b. The second flow path 4 further comprises a second side output line 4c. The second side output line 4c is arranged between the output port Lₒᵤₜ and a point P2, wherefrom the second fluid is passed into, e.g., a diluted fluid container (see FIG. 5, ref. 16) or directly for further mixing. Hence, the second side output line 4c fluidly connects the output port Lₒᵤₜ and a collection container or provides the diluted concentrate for further mixing directly. The concentrate pump 10 is positioned and arranged to provide a flow from the source of second fluid, e.g., the concentrate container 15 to the second side 2b and further through the second side 2b and the second side output line 4c to the diluted fluid container. In FIG. 1, the first flow path 3 is arranged for passing a first fluid via the first side 2a, while the second flow path 4 is arranged for passing a second fluid via the second side 2b. In some embodiments, a flow sensor 45 is positioned and arranged to sense the flow rate of the diluted concentrate fluid outputted from the second side 2b. The flow sensor 45 is connected to second side output line 4c.

The geometry of the FO-membrane 2c is here hollow fiber. The FO-membrane 2c is a water permeable membrane. The FO-membrane 2c is designed to be more or less exclusively selective towards permeating water molecules, which enables the FO-membrane 2c to separate water from all other contaminants. The FO-membrane 2c typically has a pore-size in the nanometer (nm) range, for example, from 0.5 to 5 nm or less depending on the solutes that are intended to be blocked. Suitable FO-units for FO-unit 2 may be provided by, e.g., Aquaporin™, AsahiKASEI™, Berghof™, CSM™, FTSH₂O™, Koch Membrane Systems™, Porifera™, Toyobo™ and Toray™.

A control arrangement 60 is arranged to control the apparatus 1 to perform a plurality of procedures. The control arrangement 60 includes a control unit 30, a valve arrangement 20 (20a-20p) and at least one pump 6, 7, 10. The valve arrangement 20 is positioned and arranged to configure a plurality of different flow paths of the apparatus 1. In some embodiments, the control arrangement 60 is configured to control the apparatus 1 to perform a procedure, or steps of a procedure, for diluting a dialysis concentrate and producing a dialysis fluid. The control unit 30 may comprises at least one memory and at least one processor. The at least one memory includes computer instructions for performing a procedure, or steps of a procedure, for diluting a dialysis concentrate and producing a dialysis fluid. When executed on the at least one processor, the control unit 30 controls the at least one pump and/or one or more valves 20 of the apparatus 1 to perform the one or more procedures as described herein.

The apparatus 1 is also arranged for performing one or more priming procedure(s) on the FO-unit 2. Therefore, the apparatus 1 comprises a return path 8 and the gas collection chamber 5. The priming fluid used for the one or more priming procedures is the fluid provided at the point P1, for example spent dialysis fluid or water from the container 19 or provided otherwise at point P1. Hence, the priming fluid may be the same feed fluid that is used during production for diluting a dialysis concentrate. The control arrangement 60 is also arranged to control the apparatus 1 to perform the one or more priming procedure(s), which is explained in detail herein. The return path 8 is arranged between the drain line 3d and the first side input line 3a. Hence, the return path 8 fluidly connects the drain line 3d and the first side input line 3a. The return path 8 may include one or more line(s). The return path 8 is connected to the drain line 3d between the second pump 7 and the drain valve 20i. The return path 8 is further connected to the first side input line 3a at a point between a connection point of the direct flow line 3e to the first side input line 3a and the inlet port 5a of the gas collection chamber 5. Hence, the apparatus 1 comprises a return path 8 fluidly connecting the inlet port Eᵢₙ of the first side 2a to the outlet port Eₒᵤₜ of the first side 2a of the FO-unit 2. A return path valve 20c is connected to the return path 8 to regulate a flow in the return path 8. When the apparatus 1 is producing a diluted dialysis concentrate, the return path valve 20c is closed, and the return path 8 is not in use. In some embodiments, a gas bubble sensor 44 is connected to the return path 8 to detect presence of gas bubbles such as air bubbles in the return path 8. Further, the gas collection chamber 5 is arranged in the first flow path 3 between the first side 2a and the return path 8. In some embodiments, the apparatus 1 comprises a gas collection path 9 for removing gas from the gas collection chamber 5. The gas collection path 9 is arranged between a gas outlet port 5c of the gas collection chamber 5 and the drain line 3d. Hence, the gas collection path 9 fluidly connects the gas outlet port 5c and the drain line 3d. The gas collection path 9 connects to the drain line 3d downstream of the second pump 7 and downstream of the connection point of the return path 8 to the drain line 3d. Hence, the apparatus 1 comprises a gas collection path 9 that fluidly connects the gas outlet port 5c of the gas collection chamber 5 to a drain 31 (FIG. 5). A gas collection path valve 20n is connected to the gas collection path 9 to regulate the flow in the gas collection path 9.

The return path 8 allows priming fluid expelled from the outlet port Eₒᵤₜ to circulate to the inlet port Eᵢₙ via the return path 8. Hence, the return path 8, the gas collection chamber 5 and the first side 2a are all included in a recirculation path 12. The recirculation path 12 also includes the connection line 3c, part of the first side input line 3a and part of the drain line 3d. By closing drain valve 20i, gas collection path valve 20n, direct flow line valve 20s and first side input line valve 20b, opening return path valve 20c and operating the second pump 7, priming fluid present in the recirculation path 12 is recirculated in the recirculation path 12. In some embodiments, the second pump 7 is configured to be operated in two directions, a first direction (here forwards) and a second direction (here backwards). The second pump 7 is then arranged to provide flows in opposite directions in the recirculation path 12. The second pump 7 is for example a volumetric or non-volumetric pump that can operate in both directions, hence, it is bi-directional. In the case in which second pump 7 is a volumetric pump, it may be operated in open loop (certain voltage or frequency command from control arrangement 50 to provide a certain flow rate). In the case in which the second pump is a non-volumetric pump, it may be operated with feedback from a flow sensor 41 to reach a certain flow rate (in FIG. 1 the flow sensor 41 is connected to return path 8 but may be connected anywhere to the recirculation path 12) or with feedback from one or more pressure sensors 42a, 42b to reach a certain pressure, wherein at least one sensor is connected to the recirculation path 8 such that the sensor can sense the pressure of the fluid inputted to the first side 2a. In FIG. 1, a first pressure sensor 42a is connected to the recirculation path 8 between the second pump 7 and the inlet port Eᵢₙ to sense the pressure when fluid is pumped in the first direction, and a second pressure sensor 42b is connected to the recirculation path 8 between the second pump 7 and the outlet port Eₒᵤₜ to sense the pressure when fluid is pumped in the second direction.

FIG. 2 illustrates a schematic of the gas collection chamber 5 in FIG. 1 according to some embodiments of the present disclosure. The gas collection chamber 5 is for example a deaeration chamber, a drip chamber or an air trap. The gas collection chamber 5 comprises a wall segment 5d that encloses a volume 55. The wall segment 55 for example has a cylindrical shape with a top portion and a bottom portion (e.g., like a can). The wall segment 5d is provided with the inlet port 5a, the output port 5b and the gas outlet port 5c. These ports in one embodiment are the only connections between the volume 55 and the exterior of the gas collection chamber 5. When connected within the apparatus 1, the ports are connected to fluid paths or lines as previously explained. The inlet port 5a is typically arranged at a higher level than the outlet port 5b to facilitate gas release from the priming fluid in the gas collection chamber 5 before the priming fluid is passed out of the outlet port 5b. However, priming fluid may instead be inputted into the gas collection chamber 5 via the outlet port 5b and outputted via the inlet port 5a, while gas in the fluid is nevertheless released in the gas collection chamber 5. The inlet port 5a and the outlet port 5b are typically arranged at opposite sides of the wall segment 5d of the gas collection chamber 5. One or both of the ports 5a, 5b may be tangentially arranged in the wall segment 5d. The fluid introduced via a tangentially arranged port creates a swirl that promotes separation of gas from the fluid by keeping the fluid close to the wall segment 5d and the gas in the gas collection chamber's 5 central area. The swirl also holds the fluid close to the fluid ports 5a, 5b to ensure that there is fluid at the port where the fluid shall be expelled from the gas collection chamber 5. Hence, the gas collection chamber 5 comprises at least two fluid ports 5a, 5b, wherein the priming fluid is allowed to enter and to be expelled via any of the at least two ports. The gas outlet port 5c is arranged for evacuating gas from the gas collection chamber 5. The gas outlet port 5c is typically arranged in an uppermost part of the wall segment 55, for example in the top portion. In some embodiments, the gas collection path 9 fluidly connects the gas outlet port 5c of the gas collection chamber 5 to a drain. Gas is thereby accumulated in the gas collection chamber 5 and can be removed from the gas collection chamber 5 in an easy manner. A level sensor arrangement 22 is positioned and arranged to measure a level 35 of fluid in the gas collection chamber 5. In one embodiment, the level sensor arrangement 22 comprises two sensors, an upper sensor arranged above a lower sensor. The two sensors indicate if they sense fluid or not. The fluid level 35 should typically be located between the two sensors, so the level is too low if none of the sensors indicate sensed fluid. Fluid level 35 is appropriate if the lower sensor senses fluid but the upper sensor does not. The fluid level is too high if both sensors indicate that they sense fluid. The level sensor arrangement 22 is configured to send sensed values or outputs to the control unit 30 of the control arrangement 60, which is configured to receive the sensed values or outputs and monitor the level based thereon. Based on the monitoring, the gas collection chamber 5 may be automatically filled such that the fluid level becomes appropriate.

The at least one memory of the control unit 30 stores computer instructions to perform the one or more priming procedure(s). When the at least one processor of the control unit 30 executes the instructions, the at least one pump 6, 7, 10 and the valve arrangement 20 are controlled to perform the one or more priming processes. For example, the control unit 30 may be configured to send one or more control signal(s) or control data to the at least one pump 6, 7, 10 and valves of the valve arrangement 20. A pump may be controlled to a certain speed corresponding to a certain flow rate. Generally, a valve connected to a line or path may be an on/off valve. When the valve is open, fluid flow in the line or path is allowed, and when it is closed, a fluid flow in the line or path is stopped. Alternatively, a valve may be a control valve that can be controlled to allow a certain flow rate between zero flow and unimpeded flow.

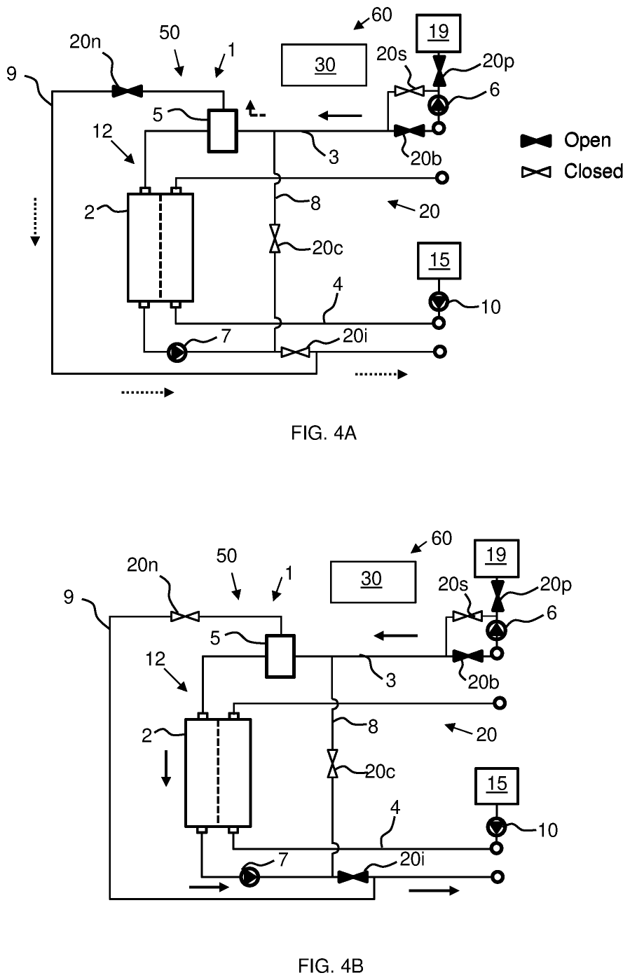

To perform the one or more procedures, the control arrangement 60 is configured to perform a plurality of measures. For example, the control arrangement 60 is configured to perform the method as explained in the flowchart in FIG. 3, which is explained as follows. In some embodiments, the control arrangement 60 is configured to perform one or more of the following:
provide priming fluid from a priming fluid source to the first flow path 3. In some embodiments, the first pump 6 is positioned and arranged to provide the priming fluid to the first path 3 from the priming fluid source. The priming fluid source is for example the container 19 or other source connected to point P1. The control unit 30 then sends control signals/data to the first pump 6 to have a certain speed providing a certain flow rate or pressure. Also, the control unit 30 controls appropriate valves to provide the fluid.
monitor a level of priming fluid in the gas collection chamber 5 and provide priming fluid to the gas collection chamber 5 until the level of priming fluid in the gas collection chamber 5 has reached a predetermined level. For example, sensed signals or output from level sensing arrangement 22 are sent to or collected by the control unit 30, which controls the apparatus 1 based on the sensed signals.
evacuate gas from the gas collection chamber 5 while providing priming fluid to the gas collection chamber 5. The control unit 30 for example opens the gas collection path valve 20n to let the gas out along the gas collection path 9.
provide priming fluid from the priming fluid source to the first side 2a of the FO-unit 2 via the first flow path 3 and the gas collection chamber 5 to fill the first side 2a with priming fluid. The control unit 30 then for example closes the gas collection path valve 20n.
provide priming fluid from the priming fluid source to the first side 2a of the FO-unit 2 upon the level of priming fluid in the gas collection chamber 5 reaching a predetermined level.
stop flow via the return path 8 while priming fluid is provided to the first flow path 3 from the container 19, here the priming fluid source. The control unit 30 then closes the return path valve 20c.
circulate the priming fluid provided to the first side 2a in a first direction in a recirculation path 12. In some embodiments, the recirculation path 12 is defined to include the first side 2a, the return path 8 and the gas collection chamber 5. In some embodiments, the second pump 7 is positioned and arranged to circulate the priming fluid in the recirculation path 12. The control unit 30 then opens return path valve 20c, closes first side input line valve 20b, directs flow line valve 20s and drain valve 20i, and controls second pump 7 to a certain speed to provide a certain flow rate or pressure.
circulate the priming fluid in a second direction in the recirculation path 12. The control unit 30 then controls second pump 7 to a certain speed in the second direction to provide a certain flow rate or pressure.
repeat (i) circulation of priming fluid in the first direction in the recirculation path 12 and (ii) circulation of the priming fluid in the second direction in the recirculation path, e.g., at least one time, until one or more predetermined priming criteria is/are fulfilled.
provide fluid from a solution source 15 via the second flow path to the inlet port Lᵢₙ to the second side 2b upon fulfilling the one or more predetermined priming criteria. In some embodiments, the inlet port Lᵢₙ is arranged below the outlet port Lₒᵤₜ of the second side 2b. The control unit 30 then controls concentrate pump 10 to a certain speed to provide a certain flow rate or pressure.
circulate the priming fluid in the first direction at a first flow rate and circulate the priming fluid in the second direction at a second flow rate, wherein the first flow rate is different from the second flow rate. In some embodiments, the first flow rate is greater than the second flow rate.
circulate the priming fluid in the first direction in the recirculation path 12 for a predetermined first time period and circulate the priming fluid in the second direction in the recirculation path 12 for a second time period, wherein the first time period and the second time period have different lengths. In some embodiments, the first time period is greater than the second time period.

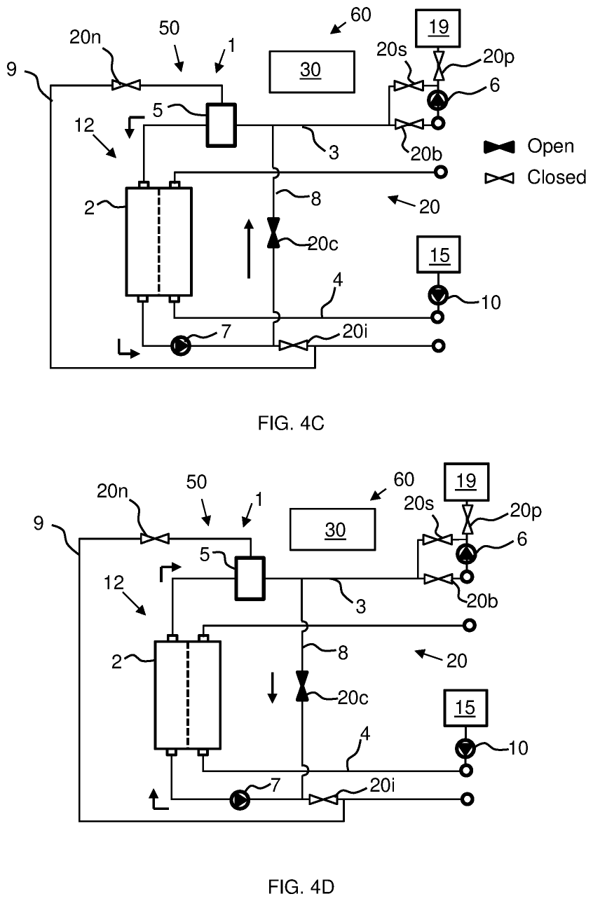

Methods for priming a FO-unit 2 will now be explained with reference to the flow chart of FIG. 3 and FIGS. 4A-4E illustrating different steps of the priming. Opened valves are shown as darkened, filled valves, and closed valves are non-filled, in accordance with the legend shown in the figures, to help identify the current flow path. In FIGS. 4A-4E certain reference numbers have been removed to clarify the steps, but are nonetheless included with all structure, functionality and alternatives discussed in connection with FIGS. 1 and 5. Also, certain components in FIG. 1 such as pressure sensors, flow sensors and gas bubble detector are omitted from FIGS. 4A-4E and 5 to make those figures more clear, but it should be understood that each of such omitted components may also be provided in FIGS. 4A-4E and 5, including all structure, functionality and alternatives discussed in connection with FIG. 1. The method is typically stored as a computer program on a computer-readable medium such as on the one or more memory of control unit 30 of the apparatus 1. The computer program includes instructions to cause the apparatus 1 to operate as described according to any one of the embodiments herein to execute the method according to any one of the embodiments described herein. When the instructions are executed by one or more processor of control unit 30 of the apparatus 1, one or more processes for priming the FO-unit 2 are performed.

The FO-unit 2 is for example the FO-unit in any of the other figures. The FO-unit 2 is maintained in the same position during the priming as illustrated in the figures, which is the same as when performing a process of diluting a dialysis concentrate, as previously explained. In this position, when performing a process of diluting a dialysis concentrate, a first fluid is introduced at the inlet port Eᵢₙ and expelled at the outlet port Eₒᵤₜ of the first side 2a, and a second fluid is introduced at the inlet port Lᵢₙ and expelled at the outlet port Lₒᵤₜ of the second side 2b.

The proposed method may be used to remove gas from the first side 2a according to a predetermined routine, e.g., at predetermined occasions throughout a recurring 24h treatment cycle. The method is performed for example by the explained control arrangement 60 by controlling valves of the valve arrangement 20, controlling one or more pumps 6, 7, 10, monitoring levels, etc. The control arrangement 60 provides appropriate control signals to the valves and pumps and receives operation data and other data such as level data to perform the method. The method may be performed before each use of the apparatus 1, hence, before each time a process of diluting a dialysis concentrate is started. Alternatively, or in combination, an assessment of the current first side 2a priming status may reveal when the method should be performed. For example, the method may be performed upon the level in the gas collection chamber 5 being too low. A considerable amount of air may then have been added via the first fluid source, which could have entered the first side 2a and thereby have altered the water extraction performance. Alternatively, the method may be performed upon obtaining an unexpected high conductivity and/or unexpected low flow rate of the fluid outputted from the second side 2b, considering the current operating point and recent operating history. Hence, the priming may be performed during production, as a response to, e.g., a reduced performance of the apparatus (e.g., increased conductivity of diluted concentrate fluid measured with conductivity sensor 11, FIG. 5, or reduced flow rate of diluted concentrate fluid measured with flow sensor 45 connected to second side output line 4c). The measured conductivity is compared with an expected conductivity, and in response to that the measured conductivity being greater than the expected conductivity, priming is initiated. The expected conductivity may be a conductivity value, threshold or interval. Alternatively, or in combination, the flow rate of diluted concentrate fluid is compared with an expected flow rate, and in response to the measured flow rate being lower than the expected flow rate, priming is initiated. The expected flow rate may be a flow rate value, threshold or interval. The production then has to be temporarily interrupted but can be resumed after the priming. Hence, the method may be triggered by various trigger conditions.

The method may include connecting the container line 3b to a container 19 or connecting the point P1 to source of priming fluid, before the priming starts. Alternatively, the container line 3b may already be connected to a container 19 or the point P1 connected to source of priming fluid. The method may include pumping priming fluid, using the first pump 6, from the source of priming fluid to the container 19. The container valve 20p is then open, and the first side input line valve 20b and direct flow line valve 20s are closed. The source of priming fluid is for example spent dialysis fluid from a patient or a tap of water. Priming fluid from a patient is spent dialysis fluid. The priming fluid may be the same fluid as used during production.

The method comprises providing S1 priming fluid to the first flow path 3. The priming fluid is provided from the source of priming fluid. As previously explained, the first flow path 3 comprises the gas collection chamber 5 and the first side 2a. Providing S1 priming fluid may be performed by operating the first fluid pump 6, here in a backwards direction, opening container valve 20p and first side input line valve 20b, and closing direct flow line valve 20s (as illustrated in FIG. 4A). The priming fluid is then pumped into the first side input line 3a from the container 19 and flows to the gas collection chamber 5. Alternatively, the priming fluid is pumped directly from point P1 by pumping in a forward direction with the first pump 6, opening direct flow line valve 20s and closing container valve 20p and first side input line 20b. Hence, providing S1 priming fluid includes providing priming fluid to the gas collection chamber 5. The direction of the priming fluid is indicated in FIG. 4A with a solid arrow. The priming fluid forces any gas present in the first side input line 3a to the gas collection chamber 5. In some embodiments, the method comprises evacuating gas from the gas collection chamber 5 while providing S1 priming fluid to the gas collection chamber 5. While providing S1 priming fluid to the gas collection chamber 5, drain valve 20i and the return path valve 20c are closed, to allow the level in the gas collection chamber 5 to rise and to evacuate gas present in the gas collection chamber 5. In other words, providing S1 may comprise stopping flow via the return path 8 while priming fluid is provided to the first flow path 3 from the priming fluid source. The gas may be evacuated to the exterior of the gas collection chamber 5 via a one-way valve (not shown) with leakage protection arranged to the gas outlet port 5c of the gas collection chamber 5. In some embodiments, the method comprises evacuating gas from the gas collection chamber 5 to the drain via the gas collection path 9. The gas collection path 9 fluidly connects a gas outlet port 5c of the gas collection chamber 5 to the drain. The direction of the evacuated gas from the gas collection chamber 5 via the gas collection path 9 and the drain line 3d to drain is indicated with hatched arrows in FIG. 4A. The point P4 is thus connected to a drain. The evacuating gas to the drain is here performed by opening gas collection path valve 20n. In some embodiments, providing S1 comprises monitoring S1A a level of priming fluid in the gas collection chamber 5 and providing priming fluid to the gas collection chamber 5 until the level of priming fluid in the gas collection chamber 5 has reached a predetermined level. The predetermined level is for example a level that is between two level sensors. The gas collection chamber 5 is then for example between 50% and 90% filled. Alternatively, the predetermined level is a level when the gas collection chamber 5 is considered completely filled, which happens when both or at least the uppermost level sensor senses fluid in the gas collection chamber 5. Hence, as long as the level has not reached or is not at the predetermined level, the method comprises providing priming fluid to the gas collection chamber 5.

After reaching the predetermined level, in some embodiments, the method comprises providing priming fluid to the first side 2a, via the gas collection chamber 5, as illustrated in FIG. 4B. Hence, in some embodiments, the method comprises providing S1B priming fluid from the priming fluid source to the first side 2a of the FO-unit 2 via the first flow path 3 and the gas collection chamber 5, upon the level of priming fluid in the gas collection chamber 5 reaching a predetermined level. Providing S1B priming fluid is for example performed by operating the first fluid pump 6, here in a backwards direction, having container valve 20p, first side input line valve 20b and drain valve 20i open, and the direct flow line valve 20s, the gas collection path valve 20n and the return path valve 20c closed. In case the gas collection chamber 5 already comprises a predetermined level of priming fluid, providing S1 comprises providing the priming fluid to the first side 2a directly, via the gas collection chamber 5, without first filling the gas collection chamber 5.

In some embodiments, the pressure at the first side 2a is lowered while filling the first side 2a and/or shortly thereafter. A lowered pressure means a pressure that is lower than the atmospheric pressure. Such lowered pressure causes any gas bubbles at the first side 2a to increase in size, whereby they loosen more easily from inside the lumens. The lowered pressure also degasses the fluid at the first side 2a. The lowered pressure may be accomplished in a plurality of ways. In one alternative, the second pump 7 is a non-volumetric pump which allows leakage through the pump. When filling the first side 2a, the second pump 7 may then either not be operated or be operated to pump towards drain. In either case, air/gas and priming fluid leak through the non-volumetric second pump 7. The non-volumetric second pump 7 may act as a throttle valve. In embodiments when the second pump 7 is not operated and priming fluid is pumped towards the first side 2a with the first pump 6, the pressure at the first side 2a will not substantially change as air causes low flow resistance when passing through second pump 7. When the priming fluid reaches the second pump 7, the pressure at the first side 2a will increase as priming fluid, which causes high flow resistance when passing through second pump 7, is pushed through the second pump 7. This increase in pressure may be sensed by the first pressure sensor 42a or the second pressure sensor 42b and indicates when the priming fluid has reached the second pump 7. The second pump 7 may then be controlled to start pumping to decrease the pressure at the first side 2a to a desired low pressure. The second pump 7 is then operated with pressure feedback from the first pressure sensor 42a or the second pressure sensor 42b. Hence, a difference in pressure can be detected depending on if gas or fluid (liquid) is passing through the second pump 7. In embodiments in which the second pump 7 is being operated, and priming fluid is pumped towards the first side 2a with the first pump 6, the pressure at the first side 2a will be lower than if the second pump 7 is not being operated. Depending on the speed of the second pump 7, the pressure at the first side 2a will be lower than, equal to or greater than the atmospheric pressure. Also here, when the priming fluid reaches the second pump 7, the pressure at the first side 2a will change compared to when the second pump 7 is pumping air, and the change can be detected. Hereafter a desired low pressure can be established by operating the second pump 7 with pressure feedback from the first pressure sensor 42a or the second pressure sensor 42b. When the desired low pressure has been established, the second pump 7 is operated to maintain the lower pressure at the same value. In embodiments when the second pump 7 is a volumetric pump and priming fluid is pumped towards the first side 2a with the first pump 6, the second pump 7 is operated, otherwise it will stop flow and the first side 2a cannot be filled. To establish a low pressure at the first side 2a, the second pump 7 is operated with pressure feedback from the first pressure sensor 42a or the second pressure sensor 42b. The second pump 7 is typically operated to provide a higher flow rate than the first pump 6 for a period of time until the low pressure has been established. Thereafter the second pump 7 is operated to provide more or less the same flow rate as the first pump 6 to maintain the lower pressure at the same value. The low pressure is typically maintained by pressure feedback to the second pump 7. When a low pressure has been established, the priming fluid can be recirculated in the recirculation path 12 at the established low pressure in the whole recirculation path 12.

In the case where a low pressure at the first side 2a is not desired and the second pump 7 is a non-volumetric pump, the second pump 7 is controlled to reach a pressure at the first side 2a that is equal to or higher than the atmospheric pressure, either when the priming fluid reaches the second pump 7 or before, while priming fluid is pumped towards the first side 2a with the first pump 6. The second pump 7 may then start pumping first when the priming fluid has reached the second pump 7, which occurs for example when a predetermined amount of priming fluid has been pumped by the first pump 6 after the gas collection chamber 5 was filled or when an increased first side 2 pressure has been detected. In the case where second pump 7 is a volumetric pump, the second pump 7 is controlled to reach a pressure at the first side 2a that is equal to or higher than the atmospheric pressure while priming fluid is pumped towards the first side 2a with the first pump 6. When a pressure at the first side 2a that is equal to or higher than the atmospheric pressure has been established, the priming fluid can be recirculated in the recirculation path 12 at the established atmospheric pressure or higher.

The second pump 7 may pump any expelled priming fluid from the first side 2a to drain. In other words, in some embodiments, the method comprises providing SIB priming fluid from the priming fluid source to the first side 2a of the FO-unit 2 via the first flow path 3 and the gas collection chamber 5, in order to fill the first side 2a with priming fluid.

After filling the first side 2a, in some embodiments, the method comprises again providing S1 priming fluid to the gas collection chamber 5, while evacuating gas from the gas collection chamber 5, to fill the gas collection chamber 5 to the predetermined level. This measure may be performed as a response to checking the level in the gas collection chamber 5 with the level sensing arrangement 22. Upon the level being detected as too low, the method comprises providing S1 priming fluid to the gas collection chamber 5. Such sequence is illustrated in FIG. 4A.

When the first side 2a has been filled with priming fluid, hence, when priming fluid has reached the second pump 7 and/or when a predetermined volume of priming fluid has been pumped with the first pump 6 and optionally the first side pressure is at a low pressure and the gas collection chamber 5 has a predetermined level of priming fluid, the method comprises circulating the provided priming fluid in the recirculation path 12. This is illustrated in FIGS. 4C and 4D. For example, the method comprises operating the second pump 7 to circulate the priming fluid in the recirculation path 12. During circulation, in some embodiments, no new priming fluid is entered into the recirculation path 12. During recirculation, gas present in the recirculation path 12 that is moved around by the circulating priming fluid is collected in the gas collection chamber 5. During circulating, the return path valve 20c is open, and first side input line valve 20b, the direct flow line valve 20s, gas collection path valve 20n and drain valve 20i are closed. The container valve 20p may also be closed. The method comprises circulating S2 the provided priming fluid in a first direction in the recirculation path 12. The second pump 7 is then operated to provide a flow of priming fluid in a first direction. In some embodiments, circulating S2 priming fluid in the first direction means circulating fluid such that it is inputted at the inlet port Eᵢₙ and outputted via the outlet port Eₒᵤₜ of the first side 2a, hence in the direction indicated by the arrows in FIG. 4C. The first direction through the first side 2a corresponds to the direction of the first fluid during production. Circulating S2 typically comprises circulating the provided priming fluid in the first direction for a predetermined time period. The predetermined time period is for example between 10 and 120 seconds. As gas is collected in the gas collection chamber 5, the level of priming fluid in the gas collection chamber 5 decreases. In some embodiments, the flow rate when circulating the priming fluid for the first time is a low flow rate, for example 200-400 ml/min. In another embodiment, the flow rate is high, for example 400-1500 ml/min. However, flow rates depend on system component dimensions, type of FO-membrane, etc., and thus may vary.

In some embodiments, the method comprises maintaining a low pressure at the first side 2a during circulation of the priming fluid in the recirculation path 12. Priming may thereby become more effective. A low pressure may be established by operating the first pump 6 and the second pump 7 to achieve different flow rates prior to entering the recirculation phase.

After circulating the priming fluid for the first time, the method in some embodiments comprises providing S1 priming fluid to the gas collection chamber 5 one more time, while evacuating gas from the gas collection chamber 5, to fill the gas collection chamber 5 to the predetermined level. This measure may be performed as a response to checking the level in the gas collection chamber 5 with the level sensing arrangement 22. Upon the level being too low, the method comprises providing S1 priming fluid to the gas collection chamber 5. As explained, this sequence is illustrated in FIG. 4A.

The recirculation path 12, and thus the first side 2a of the FO-unit 2, have now been relieved of gas that can be relatively easily removed. However, the first side 2a may still contain gas that has become trapped. To remove such trapped gas, in some embodiments, the method comprises circulating the priming fluid at a high flow rate such as 400-1500 ml/min through the recirculation path 12. Also, in some embodiments, the method comprises circulating the priming fluid in opposite directions through the recirculation path 12. Hence, the method may also comprise circulating S3 the priming fluid in a second direction in the recirculation path 12. In some embodiments, circulating S3 priming fluid in the second direction means to circulate fluid such that it is inputted at the outlet port Eₒᵤₜ and outputted via the inlet port Eᵢₙ of the first side 2a, hence in the direction indicated by the arrows in FIG. 4D that is opposite the first direction. The second pump 7 then is operated to provide a flow of priming fluid in a second direction. In some embodiments, the method comprises circulating the priming fluid in opposite directions through the recirculation path 12, at high flow rate. For providing different flow rates, the second pump 7 is operated at different speeds. In some embodiments, the method comprises circulating the priming fluid in different directions through the recirculation path 12, at both a high flow rate, e.g., 400-1500 ml/min and a low flow rate, e.g., 50-200 ml/min or 200-400 ml/min. In some embodiments, the method comprises circulating S2 the priming fluid in the first direction at a first flow rate and circulating S3 the priming fluid in the second direction at a second flow rate, wherein the first flow rate is different from the second flow rate. In some embodiments, the first flow rate is greater than the second flow rate. The circulating in different directions may follow immediately after each other, or very shortly after each other. In some embodiments, the method comprises circulating the priming fluid in different directions for different length of time periods. In some embodiments, the method comprises circulating S2 the priming fluid in the first direction in the recirculation path 12 for a predetermined first time period and circulating S3 the priming fluid in the second direction in the recirculation path 12 for a second time period, wherein the first time period and the second time period have different lengths. The first time period is for example greater than the second time period. Any of the above steps may be combined and/or repeated one or more time(s). In some embodiments, the method comprises (i) circulating S2 the priming fluid in the first direction in the recirculation path 12 and (ii) circulating S3 the priming fluid in the second direction in the recirculation path 12, at least one time until one or more predetermined priming criteria is/are fulfilled. For example, in some embodiments, the method comprises circulating the priming fluid in the first direction with a high flow rate for a first time period. The first time period is for example between 10 and 120 seconds. This step is performed to force any gas present in the fiber lumens at the first side 2a out from the lumens by means of a high pressure drop caused by the high flow rate. Hence, the high flow rate causes a high pressure drop from the inlet to the outlet at the first side 2a because of higher flow resistance over the first side 2a. The circulation is then stopped. In the example, the method thereafter comprises circulating the priming fluid in the second direction with a low flow rate for a second time period. The purpose of circulating the priming fluid in the second direction is to transport gas present close to the inlet port Eᵢₙ to the gas collection chamber 5. The second time period should be long enough to allow gas in the uppermost part of the first side 2a to be transported with the priming fluid from the first side 2a to the gas collection chamber 5. Thus, the second time period depends on the length of the connection line 3c and on the magnitude of the low flow rate. For example, the second time period is some number of seconds, for example 3 to 10 seconds. The first time period is typically longer than the second time period, as the priming fluid at the first side 2a has to travel a longer distance to reach the gas collection chamber 5 when circulated in the first direction, than the priming fluid at the first side 2a when circulated in the second direction. Hence, the length of the first time period is typically at least the time it takes for the priming fluid at the first side 2a to travel to the gas collection chamber 5 via the return path 8. The procedure of circulating the priming fluid in the first direction with a high flow rate for a first time period, and thereafter circulating the priming fluid in the second direction with a low flow rate for a second time period, may be performed a plurality of times. For example, the procedure may be repeated 5 to 10 times to ensure a gas-free first side 2a. Hence, a criterion to stop the repeating or circulating may be that the repeating or circulating has been performed a plurality of times for example a certain number of times. In some embodiments, the apparatus 1 comprises the gas bubble sensor 44 connected to the return path 8. The method may then include the detection of a presence of gas bubbles based on, e.g., gas bubble size and/or number. In response to detection of gas bubbles satisfying one or more low detection criteria, the repeating or circulating may be stopped and the priming of the first side be considered satisfactory. The low detection criteria may include for example no gas bubbles larger than a predetermined size for a predetermined time period, and/or less than a predetermined number of gas bubbles that are greater than a predetermined size during a predetermined time period.

In some embodiments, in addition to circulating the priming fluid in the first direction with a high flow rate for a first time period, and thereafter circulating the priming fluid in the second direction with a low flow rate for a second time period, the method includes circulating the priming fluid in the second direction at a high flow rate through the first side 2a. More gas bubbles may accordingly be removed from the fiber lumens and collected in the gas collection chamber 5. Circulating the priming fluid in the second direction at a high flow rate through the first side 2a is performed for a third time period. The third time period follows after the second time period, typically directly after the second time period. The third time period may have the same length as the second time period, hence between 3 to 10 seconds.

The first side 2a has now been primed and is presumably gas-free, at least free from larger gas bubbles. This also means that the one or more predetermined priming criteria has been fulfilled for the first side 2a. In some embodiments, upon the one or more predetermined priming criteria being fulfilled, the method comprises providing fluid from a solution source 15 via a second flow path to the inlet port Lᵢₙ to the second side 2b which is arranged below the outlet port Lₒᵤₜ of the second side 2b. Alternatively, the providing fluid from a solution source 15 to the second side 2b is performed before, during and/or after the priming of the first side 2a. The fluid from the solution source 15 is typically a concentrate solution that is to be provided to the second side 2b during dialysis fluid production. Hence, the priming of the second side 2b may also be a start of the process of diluting the concentrate. The second side 2b is filled from bottom and up as illustrated in FIG. 4E, and any gas at the second side 2b is transported by the diluted concentration solution to a collection chamber (FIG. 5, ref 16). As the second side 2b is located along the outside of the lumen (shell side), gas does not get trapped that easily at the second side 2b. After the second side 2b is filled, the priming of the FO-unit 2 is completed. The FO-unit 2 can now efficiently produce a diluted dialysis concentrate fluid. In FIG. 4E it is illustrated that a feed fluid flows at the first side 2a and a draw fluid flows at the second side 2b.

The higher pressure at the first side 2a (than the lower pressure at the first side 2a during priming) is typically associated with a water extraction session that reduces the size of any remaining and potentially flow obstructing gas bubbles. The session also decreases the risk of additional gas formation due to fluid degassing.

In addition to the above-described measures, in some embodiments, flow and/or pressure transients may be used to force gas bubbles out of the fiber lumens at the first side 2a. Hence, the method may include providing a pulsating flow creating flow transients that aid in gas bubble release from the fiber lumens at the first side 2a. The method may then include operating the second pump 7 to provide a pulsating flow or periodic flow pattern in the recirculation path 12, hence while the priming fluid is recirculated as illustrated in FIG. 4C or 4D. Alternatively, or in combination, the method may include creating a built-up pressure that is periodically released to create large flow rate pulses. This will create flow transients that aid in gas bubble release from the fiber lumens. For example, the first pump 6 may periodically build a pressure in the gas collection chamber 5 and then release it through the first side 2a of the FO-unit 2.

In some embodiments, the priming is enhanced by exerting the FO-unit 2 for motion by external means to facilitate air removal, for example mechanically vibrating the FO-unit 2.

One example priming scenario is now explained with reference to the FIGS. 1 and 4A-4F. As a response to a predetermined occasion, e.g., each day at 1 PM, a priming sequence is started. The priming sequence starts by filling the gas collection chamber 5 with priming fluid up to a predetermined level, while gas is evacuated via the evacuation path 9, see steps S1-S1A and FIG. 4A. Thereafter the first side 2a is filled by providing a predetermined amount of priming fluid to the first side 2a, see step S1B and FIG. 4B. The gas collection chamber 5 is thereafter again filled with priming fluid up to the predetermined level, while gas is evacuated via the evacuation path 9, see steps S1-S1A and FIG. 4A. The priming fluid in the recirculation path 12 is thereafter recirculated in the recirculation path 12 in the first direction, hence, a forward direction, see step S2 and FIG. 4C. The gas collection chamber 5 is thereafter again filled with priming fluid again up to the predetermined level, while gas is evacuated via the evacuation path 9, see steps S1-S1A and FIG. 4A. A priming sequence is now repeatedly performed, including circulating the priming fluid in the first direction with a high flow rate for a first time period, followed by circulating the priming fluid in the second direction with a lower flow rate for a second time period, see steps S2-S4 and FIGS. 4C and 4D. The sequence may be repeated, e.g., 5-10 times, to ensure an air-free feed side. Thereafter the first side 2a is primed, see FIG. 4E. A concentrate fluid is now provided to the second side 2b to fill the second side 2b, see step S5. When the second side 2b is filled, the second side 2b is also primed, and the whole FO-unit 2 is considered primed. Hence, first the first side 2a is primed while the second side 2b is empty. Thereafter the second side 2b is primed, while the first side 2a is already filled, that is, has already been primed. Alternatively, the second side 2b is primed before or while the first side 2a is primed.

An example of an apparatus 1 for producing fluid for dialysis will now be explained with reference to FIG. 5. The legend regarding open and closed valves does not apply to FIG. 5. The apparatus 1 comprises the part 50 illustrated in FIG. 1 and FIGS. 4A to 4E, together with some additional components that were left out from FIG. 1. Hence, the apparatus 1 comprises a FO-unit 2, a first flow path 3 and a second flow path 4. The explained methods for priming can equally be applied to the apparatus 1 in FIG. 5.

The first flow path 3 starts at an inlet connector Pi and ends at the drain 31. The second flow path 4 starts at the concentrate container 15 and ends at outlet connector Po. The inlet connector Pi is for example connectable to a catheter of a PD patient, or to a spent dialysis fluid line of a HD or CRRT apparatus. The outlet connector Po is for example connectable to a catheter of a PD patient, or to a dialysis fluid line of a HD or CRRT apparatus. The first flow path 3 comprises a plurality of fluid lines, namely, a first side input line 3a, a container line 3b, a connection line 3c and a drain line 3d. The first side input line 3a is arranged between the input point Pi and an inlet port 5a of a gas collection chamber 5, for example, a gas collection chamber 5 as illustrated in FIG. 2. The first side input line 3a fluidly connects the input point Pi and the inlet port 5a of a gas collection chamber 5. The container line 3b is arranged between the container 19 and a connection point P1 of the first side input line 3a. As illustrated, the connection point P1 in FIGS. 1 and 4A-4E corresponds to the connection point P1 in FIG. 5. The container line 3b connects the container 19 and the first side input line 3a. The connection line 3c is arranged between the outlet port 5b (FIG. 2) of the gas collection chamber 5 and the inlet port Eᵢₙ of the first side 2a. Hence, the connection line 3c fluidly connects the outlet port 5b of the gas collection chamber 5 and the inlet port Eᵢₙ. The drain line 3d is arranged between the outlet port Eₒᵤₜ of the first side 2a and connection point P4 that is connected to a drain 31. Hence, the drain line 3d fluidly connects the outlet port Eₒᵤₜ and the drain 31. A pressure sensor 26 is connected to the first side input line 3a to sense the pressure of the fluid in the first side input line 3a. The sensed pressure from the pressure sensor 26 is representative of the pressure in the gas collection chamber 5. A first pump 6 is arranged to operate with the container line 3b, to provide a flow in the container line 3b. The first pump 6 is positioned and arranged to pump fluid in a forward direction to fill the container 19 with, e.g., spent dialysis fluid. The first pump 6 is also arranged to pump fluid in a backward direction to pump fluid from the container 19 in the direction to the gas collection chamber 5. An input valve 20a is arranged to operate with the first side input line 3a between the inlet connector Pi and the point P1. A first side input line valve 20b is arranged to operate with the first side input line 3a between the point P1 and the gas collection chamber 5. A second pump 7 is arranged to operate with the drain line 3d to provide a flow of fluid in the drain line 3d. A drain valve 20i is arranged to operate with the drain line 3d between a connection point of the return path 8 to the drain line 3d and a connection point of the gas collection path 9 to the drain line 3d. A return path 8 is arranged between the drain line 3d and the first side input line 3a. Hence, the return path 8 fluidly connects the drain line 3d and the first side input line 3a. The return path 8 is connected to the drain line 3d between the second pump 7 and the drain valve 20i. The return path 8 is further connected to the first side input line 3a at a point between a connection point of the direct flow line 3e to the first side input line 3a and the inlet port 5a of the gas collection chamber 5. A gas collection path 9 is arranged between a gas outlet port 5c (FIG. 2) of the gas collection chamber 5 and the drain line 3d. Hence, the gas collection path 9 fluidly connects the gas outlet port 5c and the drain line 3d. The gas collection path 9 connects to the drain line 3d between the drain valve 20i and the drain. Spent dialysis fluid may be collected in the container 19 by pumping spent dialysis fluid to the container 19 with the first pump 6, opening first input valve 20a and container valve 20p, and closing first side input line valve 20b and direct flow line valve 20s. Spent dialysis fluid can thereafter be transported to the first side 2a by pumping with the first pump 6 and the second pump 7, opening first side input line valve 20b, container valve 20p and drain valve 20i, and closing inlet valve 20a, direct flow line valve 20s, return path valve 20c and gas collection path valve 20n. The gas collection chamber 5 may be filled by additionally opening gas collection path valve 20n and not operating second pump 7 or operating it at a lower flow rate than the first pump 6. The first side 2a may be filled by additionally closing gas collection path valve 20n.

The second flow path 4 comprises a plurality of fluid lines, namely a concentrate line 4d, a second side input line 4b, a second side output line 4c, a first diluted concentrate line 4e, a second diluted concentrate line 4a, a main line 4f, a pure water line 4g, a second concentrate line 4h and a drain connection line 4i. The concentrate line 4d is arranged between a concentrate container 15 and a connection point P3 to the main line 4f and to the second side input line 4b. Hence, the concentrate line 4d connects the concentrate container 15 to the second side input line 4b (and to the main line 4f). As illustrated, the connection point P3 in FIGS. 1 and 4A-4E corresponds to the connection point P3 in FIG. 5. A concentrate valve 20d is connected to the concentrate line 4d. The second side input line 4b is arranged between the connection point P3 to the concentrate line 4d and the inlet port Lᵢₙ of the second side 2b. Hence, the second side input line 4b connects the connection point P3 and the inlet port Lᵢₙ. A concentrate pump 10 is positioned and arranged to provide a flow in the concentrate line 4d. A second side input valve 20h is connected to the second side input line 4b. The second side output line 4c is arranged between the outlet port Lₒᵤₜ of the second side 2b and a connection point P2 on the first diluted concentrate line 4e. As illustrated, the connection point P2 in FIGS. 1 and 4A-4E corresponds to the connection point P2 in FIG. 5. Hence, the second side output line 4c connects the outlet port Lₒᵤₜ and the first diluted concentrate line 4e. The first diluted concentrate line 4e is arranged between an inlet of the diluted fluid container 16 and the connection point P3 at the concentrate line 4d. Hence, the first diluted concentrate line 4e connects the inlet of the diluted fluid container 16 and the connection point P3 of the concentrate line 4d. A first diluted concentrate valve 20e is connected to the first diluted concentrate line 4e between the connection point P2 of the second side output line 4c to the first diluted concentrate line 4e, and the connection point of the first diluted concentrate line 4e to concentrate line 4d. A conductivity sensor 11 is connected to the first diluted concentrate line 4e between the point P2 and the inlet of the diluted fluid container 16. The main line 4f is arranged between the connection point P3 to the concentrate line 4d, and the outlet connector Po. Hence, the main line 4f connects the connection point P3 and the outlet connector Po. The second diluted concentrate line 4a is arranged between an outlet of the diluted fluid container 16 and the connection point P3 to the main line 4f. A second diluted concentrate valve 20f is connected to the second diluted concentrate line 4a. Hence, the connection point P3 connects the main line 4f, the concentrate line 4d, the second diluted concentrate line 4a and the second side input line 4b. The second flow path 4 further comprises a plurality of components connected to the main line 4f, namely, a main valve 20g, a heating element 65, a temperature sensor 27, a main pump 23, a mixing chamber 24, a conductivity sensor 25 and an outlet valve 20j. A pure water line 4g is arranged between a pure water container 17 and the main line 4f. Hence, the pure water line 4g connects the pure water container 17 and the main line 4f. The main valve 20g is connected to the main line 4f between the point P3, and the connection point of the pure water line 4g to the main line 4f. A second concentrate line 4h is arranged between a second concentrate container 18 and the main line 4f. Hence, the second concentrate line 4h connects the second concentrate container 18 and the main line 4f. A second concentrate pump 29 is positioned and arranged to provide a flow of second concentrate in the second concentrate line 4h. The main pump 23 is positioned and arranged to provide a flow in the main line 4f downstream the connection of the pure water line 4g to the main line 4f and downstream of the connection of the second concentrate line 4h to the main line 4f. The temperature sensor 27 is positioned and arranged to sense a temperature of the fluid in the main line 4f upstream the main pump 23, but downstream the connection of the second concentrate line 4h to the main line 4f. The mixing chamber 24 is arranged downstream the main pump 23, and upstream the main conductivity sensor 25. An exhaust valve 20m is arranged to operate with an exhaust line 4j connected between the mixing chamber 24 and the drain line 3d. The exhaust line 4j transport excessive gas in the mixing chamber 24 to drain 31.

For diluting a concentrate, the concentrate pump 10 is operated to pump concentrate solution from the concentrate container 15 to the second side 2b. The concentrate valve 20d and second side input valve 20h are then opened, and first diluted concentrate valve 20e and second diluted concentrate valve 20f are closed. Simultaneously, a spent dialysis fluid is provided at the first side 2a. Pure water will then be extracted from the spent dialysis fluid at the first side 2a to the concentrate solution at the second side 2b, by osmotic pressure difference. Thus, the concentrate solution will become diluted to form an intermediate dialysis solution, hence, a diluted concentrate solution, which is collected in diluted fluid container 16. This procedure may be referred to as a FO-session. After the diluted concentrate has been collected in the diluted fluid container 16, the diluted concentrate may be circulated in the first diluted concentrate line 4e, part of the concentrate line 4d, second diluted concentrate line 4a, and the diluted fluid container 16 by pumping with the concentrate pump 10, opening first diluted concentrate valve 20e and second diluted concentrate valve 20f, and closing second side input valve 20h, concentrate valve 20d, and main valve 20g. The conductivity sensor 11 measures the conductivity of the circulated diluted concentrate to monitor when the conductivity is stable and thus the diluted concentrate homogenous.

For mixing a dialysis fluid, the diluted concentrate solution in diluted fluid container 16 is pumped to main line 4f by operating concentrate pump 10, and opening first diluted concentrate valve 20e, main valve 20g, outlet valve 20j, and closing concentrate valve 20d, second side input valve 20h, second diluted concentrate valve 20f and drain connection valve 20k. At the same time, second concentrate solution such as glucose, is passed to the main line 4f by operating second concentrate solution pump 29. Pure water flows to the main line 4f from the pure water container 17. The main pump 23 provides a desired flow rate of resulting dialysis fluid in the main line 4f downstream main pump 23. The conductivity sensor 25 measures the conductivity of the resulting dialysis fluid from the main pump 23. The concentrate pump 10 is controlled to a certain speed to achieve a desired predetermined concentration of the resulting dialysis fluid, which is based on the conductivity of the produced fluid, the conductivity of the diluted concentrate solution, and flow rate of the produced fluid. The second concentrate solution pump 29 is controlled to a certain speed based on flow rate of the produced fluid, to achieve a certain composition of concentrate in the produced fluid. In the mixing chamber 24, the diluted concentrate solution, the second concentrate solution and the pure water are mixed to form a dialysis fluid. The mixing chamber 24 is small and may typically only accommodate 30-100 ml of fluid. Thereafter, the dialysis fluid is delivered at the outlet connector Po to a desired destination (e.g., a storage container or a dialysis machine or to a catheter connected to a PD patient). A level sensing arrangement 66 monitors the level in the mixing chamber 24 and the exhaust valve 20m is opened if the level becomes too low to in response pass gas to drain and thereby raise the level. The main conductivity sensor 25 measures the conductivity of the final dialysis fluid. If the conductivity is not within predetermined limits, the dialysis fluid is passed to drain 31 via a drain connection line 4i. A drain connection valve 20k is connected to the drain connection line 4i, which is open when dialysis fluid is passed to drain 31. A pressure sensor 28 is connected to the main line 4f downstream the output valve 20j, to sense the pressure at the outlet connector Po.

Any of the pumps described herein may for example be a volumetric pump (such as a piston pump) or a non-volumetric pump (for example a gear pump) that operates with flow rate feedback from a flow sensor (not shown). One or more of the pumps may be one-directional or bi-directional. The concentrate container 15 comprises an electrolyte solution. For example, the electrolyte solution may comprise an electrolyte and buffer, for example Na, Ca, Mg and Lactate. The second concentrate container 18 comprises for example glucose concentrate. The control arrangement 60 further comprises a control unit 30 including at least one memory and at least one processor. The control arrangement 60 is configured to control the pumps 6, 7, 10, 23 and 29 and the valves 20a-20p of the valve arrangement 20 to perform a plurality of different processes, such as to produce dialysis fluid or perform a priming process. The control arrangement 60 is also configured to receive measurements of conductivity from the conductivity sensors 11, 25. The control arrangement 60 is further configured to receive measurements of pressure from the pressure sensors 26, 28 and temperature sensor 27. Hence, the control arrangement 60 is configured to receive or collect any signal or data from the components of the apparatus 1 and to control the pumps and/or valves based thereon. The result may be provided to a user via a user interface (not shown).

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and the scope of the appended claims.

1-38. (canceled)

39. An apparatus for producing fluid for dialysis, the apparatus comprising:
a forward osmosis (FO-) unit configured for diluting a dialysis concentrate in a process for producing a dialysis fluid, the FO-unit including a FO-membrane that separates a first side from a second side of the FO-unit;
a first flow path including the first side of the FO-unit;
a control arrangement configured to provide priming fluid from a priming fluid source to the first flow path;
a return path fluidly connecting an inlet port of the first side to an outlet port of the first side of the FO-unit, the return path allowing priming fluid expelled from the outlet port to circulate to the inlet port via the return path; and
a gas collection chamber arranged in the first flow path between the first side and the return path, wherein the gas collection chamber is configured to receive gas removed from the first flow path.

40. The apparatus according to claim 39, wherein the control arrangement is configured to monitor a level of priming fluid in the gas collection chamber and to provide priming fluid to the gas collection chamber until the level of priming fluid in the gas collection chamber has reached a predetermined level.

41. The apparatus according to claim 40, which is positioned and arranged to evacuate gas from the gas collection chamber while providing priming fluid to the gas collection chamber.

42. The apparatus according to claim 39, wherein the gas collection chamber comprises a gas outlet port for evacuating gas from the gas collection chamber.

43. The apparatus according to claim 42, further comprising a gas collection path fluidly connecting the gas outlet port of the gas collection chamber to a drain.

44. The apparatus according to claim 39, wherein the control arrangement is configured to provide priming fluid from the priming fluid source to the first side of the FO-unit via the first flow path and the gas collection chamber to fill the first side with priming fluid.

45. The apparatus according to claim 44, wherein the control arrangement is configured to provide priming fluid from the priming fluid source to the first side of the FO-unit via the first flow path and the gas collection chamber, upon the level of priming fluid in the gas collection chamber reaching a predetermined level.

46. The apparatus according to claim 39, wherein the control arrangement is configured to stop flow via the return path while priming fluid is provided to the first flow path from the priming fluid source.

47. The apparatus according to claim 44, wherein the control arrangement is configured to circulate the priming fluid provided to the first side in a first direction in a recirculation path, the recirculation path comprising the first side, the return path and the gas collection chamber.

48. The apparatus according to claim 47, wherein the control arrangement is configured to circulate the priming fluid in a second direction in the recirculation path.

49. The apparatus according to claim 48, wherein the control arrangement is configured to repeat (i) circulation of the priming fluid in the first direction in the recirculation path, and (ii) circulation of the priming fluid in the second direction in the recirculation path, at least one time until one or more predetermined priming criteria is/are fulfilled.

50. The apparatus according to claim 49, further comprising a second flow path for providing fluid to the second side, wherein the control arrangement is configured, upon fulfilling the one or more predetermined priming criteria for the first side, to provide fluid from a solution source via the second flow path to an inlet port to the second side, the inlet port arranged below an outlet port of the second side.

51. The apparatus according to claim 49, wherein the control arrangement is configured to circulate the priming fluid in the first direction at a first flow rate and to circulate the priming fluid in the second direction at a second flow rate, wherein the first flow rate is different from the second flow rate.

52. The apparatus according to claim 51, wherein the first flow rate is greater than the second flow rate.

53. The apparatus according to claim 49, wherein the control arrangement is configured to circulate the priming fluid in the first direction in the recirculation path for a predetermined first time period and to circulate the priming fluid in the second direction in the recirculation path for a second time period, wherein the first time period and the second time period have different lengths.

54. The apparatus according to claim 39, wherein the control arrangement comprises at least one pump.

55. The apparatus according to claim 54, wherein the at least one pump comprises a first pump arranged to provide the priming fluid to the first path from the priming fluid source.

56. The apparatus according to claim 55, wherein the at least one pump comprises a second pump arranged to circulate the priming fluid in the recirculation path.

57. The apparatus according to claim 39, wherein the priming fluid is the same fluid that is used during production for diluting a dialysis concentrate, and wherein the priming fluid is water or used dialysis solution.

58. A method for priming a forward osmosis (FO-) unit configured to be used for diluting a dialysis concentrate in a process for producing a dialysis fluid, the FO-unit including a FO-membrane that separates a first side from a second side of the FO-unit, the method comprising:
providing priming fluid to a first flow path comprising the first side and a gas collection chamber configured to receive gas removed from the first flow path, and
circulating the provided priming fluid in a recirculation path comprising the first side, a return path and the gas collection chamber, wherein the return path fluidly connects an inlet port of the first side to an outlet port of the first side of the FO-unit.
